(19) United States
(12) Patent Application Publication
(10) Pub. No.: US 2025/0269100 A1
(43) Pub. Date: Aug. 28, 2025

(54) PERITONEAL DIALYSIS METHODS

(71) Applicant: AWAK TECHNOLOGIES PTE LTD, Singapore (SG)

(72) Inventors: Suresha BELUR VENKATARAYA, Singapore (SG); Mandar Manohar GORI, Singapore (SG); Sanjay Kumar SINGH, Singapore (SG); Joel Preetham FERNANDES, Singapore (SG); Jason Tze Chern LIM, Singapore (SG); Marcin Bartlomiej PAWLAK, Singapore (SG); Sridhar CHIRUMARRY, Singapore (SG); Vinod Kumar GADI, Singapore (SG); Yue (Victoria) WANG, Singapore (SG); Bei Ming CHEN, Singapore (SG); Siti Noor Huda JAMALUDDIN, Singapore (SG); Abel Samson DSOUZA, Singapore (SG)

(73) Assignee: AWAK TECHNOLOGIES PTE LTD, Singapore (SG)

(21) Appl. No.: 19/059,528

(22) Filed: Feb. 21, 2025

(30) Foreign Application Priority Data
Feb. 23, 2024 (SG) SG10202400503P

(51) Int. Cl. A61M 1/28

(52) U.S. Cl. CPC A61M 1/282; A61M 1/281; A61M 1/287

(57) ABSTRACT

Disclosed herein is a method of peritoneal dialysis, the method comprising the steps of: (i) administering to a subject a first hypertonic solution comprising a sugar to a peritoneal cavity in the subject, followed by; (ii) a tidal therapy phase and then a dwell phase; or a dwell phase and then a tidal therapy phase.

### FIELD OF INVENTION

The present disclosure generally relates to methods of dialysis, and more particularly relates to methods of peritoneal dialysis.

### BACKGROUND

The listing or discussion of a prior-published document in this specification should not necessarily be taken as an acknowledgement that the document is part of the state of the art or is common general knowledge.

Millions of people worldwide suffer from kidney-related problems, such as chronic kidney disease (CKD) and end-stage renal disease (ESRD), and they may require either dialysis, such as peritoneal dialysis, to maintain life. In peritoneal dialysis, the peritoneum in the patient's abdomen acts as a natural filtration membrane.

Continuous ambulatory peritoneal dialysis (CAPD) is the most common form of peritoneal dialysis. It does not require a machine, and the patient can carry out the treatment themselves. This method involves manually exchanging dialysis fluid, called dialysate, in and out of the peritoneum through a permanent catheter. The process, known as an exchange, typically occurs four times a day: morning, noon, evening, and before bed.

During CAPD, the dialysate is left in the abdomen for several hours, a period known as dwell time. During this time, waste products and excess fluid move from the blood into the dialysate through the peritoneum. After the dwell time, the dialysate, now containing the waste products, is drained out and replaced with fresh dialysate.

Continuous cycling peritoneal dialysis (CCPD, also known as automated peritoneal dialysis, APD), is similar to CAPD but uses a machine called a cycler to automate the exchanges during the night while the patient sleeps. This method allows for multiple exchanges over 8-10 hours overnight, with the last fill left in the abdomen during the day, which the patient then drains in the evening to begin the process again. CCPD is beneficial for people who have a busy daytime schedule or for children who need dialysis treatment. Tidal Peritoneal Dialysis (TPD) is a variation of CCPD that is designed to optimize dialysis efficiency and patient comfort. In TPD, the cycler automatically fills and drains the peritoneal cavity with dialysate, but unlike CCPD, it does not completely drain the dialysate with each cycle. Instead, a certain volume of dialysate always remains in the abdomen to ensure continuous dialysis. This “tidal volume” reduces pain and discomfort that some patients experience during complete drain phases. TPD can be particularly useful for patients with poor drainage or pain during exchanges.

In some modes of TPD, a small tidal volume is moved in and out of the patient frequently, and this small tidal volume is cleaned by passing through a sorbent, regenerated, and returned back to the patient.

In addition, at times, a TPD alone may not be sufficient for the removal of toxin and ultrafiltration due to difference in rate of toxin removal and ultrafiltration. For example, a patient with high transport peritoneal membrane may remove toxins at higher rate and ultrafiltration at slower rate compared to a patient with low transport characteristic of peritoneal membrane. Additional toxin removal and/or ultrafiltration can be achieved by combining TPD therapy with additional dwell of fresh/regenerated dialysate before and/or after the TPD therapy. The requirement of additional dwell may be to have a regenerated fluid after tidal therapy of tonicity similar to initial fill fluid.

The peritoneal membrane's transport characteristics are crucial for the effectiveness of Peritoneal Dialysis (PD), affecting the efficiency of solute removal and fluid exchange. Understanding these characteristics allows for personalized PD regimens that optimize treatment for individual patients.

The peritoneal membrane can be classified based on its transport characteristics, particularly how quickly it transports solutes and water (see for example, Waniewski et al., ASAIO Journal 38 (4):p 788-796, October 1992. and Waniewski et al., Blood Purif (1991) 9 (3): 129-141, which are each included herein by reference). This classification is often determined through tests like the Peritoneal Equilibration Test (PET), which measures the membrane's transport rate of solutes such as creatinine and glucose, as well as water. Based on these tests, the peritoneal membrane is typically categorized into four types:

High transporter membranes exhibit rapid solute transport and quick glucose absorption from the dialysate. This leads to a faster equilibration of solute concentrations between the blood and dialysate. While fluid removal (ultrafiltration) is initially high due to the osmotic gradient created by glucose, it can decrease over time during long dwell times because the glucose is quickly absorbed, diminishing the osmotic gradient.

High average transporters have slightly slower solute transport rates than high transporters but faster than the middle group. They still absorb glucose relatively quickly, which can impact the efficiency of ultrafiltration over longer dwell periods. This group might require a tailored approach, balancing between solute clearance efficiency and maintaining adequate fluid removal.

Low average transporters have slower solute transport rates, allowing for more extended dwell times without losing the osmotic gradient quickly. This characteristic can be advantageous for CAPD or overnight APD regimes, as it supports sustained ultrafiltration and efficient toxin removal over longer periods.

Low transporter membranes have the slowest solute transport rates. Glucose is absorbed slowly, maintaining the osmotic gradient for longer periods, which is beneficial for ultrafiltration during long dwell times. However, the slower transport rate may reduce the efficiency of solute clearance, necessitating longer or more frequent exchanges to achieve adequate dialysis.

Additionally, the peritoneal transport characteristics can change over time, especially with long-term PD therapy, which may necessitate adjustments in the PD prescription. Regular assessment of the peritoneal membrane's transport status is recommended to ensure ongoing effectiveness and safety of the PD treatment.

FIG. 1 illustrates the differences in transport rates of creatinine and glucose across peritoneal membrane for patients classified as high, high average, low average, and low transporters.

For sorbent based peritoneal dialysis, adequate clearance and water removal for various transport status can be achieved by adding optional 1-2 hour of dwell before/after tidal peritoneal dialysis without increasing the amount of sorbent cartridge and thus enabling wearability and portability of the device. The dwell period can extend from 1-2 hour and during dwell period different amount of glucose solution (Low, Medium and High setting) can be added to the patient to maintain required hypertonicity of the dwell fluid. In the sorbent phase, in addition to glucose, the calcium salt and magnesium salt are added to regenerate the fluid (see, for example, FIG. 2).

There are a few mathematical models that describe the flow of solutes and water across the peritoneal membrane during a peritoneal dialysis therapy. Some examples of these models are the three-pore model, Pyle-Popovich model, Vonesh's model, and Garred's model. These models have been used to assist physicians to prescribe conventional modes of peritoneal dialysis therapies, such as automated peritoneal dialysis (APD), continuous ambulatory peritoneal dialysis (CAPD), or tidal peritoneal dialysis (TPD). Using samples from waste dialysate drained from the patient, these models can be used to determine the mass transfer coefficient (MTAC) of the patient's peritoneal membrane, which is a measure of the solute clearance from the patient. The MTAC can be used by physicians to prescribe suitable peritoneal dialysis therapy for the patient to improve the solute clearance.

For adults on PD, the KDIGO guidelines recommend a minimum total (i.e., the sum of renal and peritoneal Kt/V) weekly Kt/V urea target of 1.7 to ensure adequate dialysis. This target considers the combined effect of residual kidney function and the dialysis process itself. The rationale behind this recommendation is to balance the need for sufficient dialysis to remove toxins and manage fluid volume while minimizing the risks associated with overly aggressive dialysis regimens, such as infections or alterations in membrane function. It is important to note that these targets may be adjusted based on individual patient needs, comorbidities, and the presence of symptoms.

### SUMMARY OF INVENTION

Aspects and embodiments of the invention are provided in the following numbered clauses
1. A method of peritoneal dialysis, the method comprising the steps of:
(i) administering to a subject a first hypertonic solution comprising a sugar to a peritoneal cavity in the subject, followed by;
(ii) a tidal therapy phase and then a dwell phase; or a dwell phase and then a tidal therapy phase;
wherein the tidal therapy phase comprises the steps of:
(a) allowing water and/or a toxin from the subject to pass into the peritoneal cavity by osmosis, thereby forming an N^{th} hypertonic solution within the peritoneal cavity;
(b) withdrawing up to about 50% by volume of the N^{th} hypertonic solution from the peritoneal cavity and combining it with a first sugar concentrate to form an N^{th}+1 hypertonic solution,
wherein the withdrawn N^{th} hypertonic solution and/or the N^{th}+1 hypertonic solution is passed through a dialysis sorbent, followed by adding calcium and magnesium ions into the withdrawn N^{th} hypertonic solution and/or the N^{th}+1 hypertonic solution;
(c) administering the N^{th}+1 hypertonic solution to the peritoneal cavity to form an N^{th}+2 hypertonic solution within the peritoneal cavity by mixing of the N^{th} and N^{th}+1 hypertonic solutions; and
(d) repeating steps (a) to (c) every 5 minutes to 30 minutes for a treatment time of about 5 hours to about 10 hours; and
wherein the dwell phase comprises the steps of:
(A) allowing water and/or a toxin from the subject to pass into the peritoneal cavity by osmosis, thereby forming an X^{th} hypertonic solution within the peritoneal cavity;
(B) withdrawing up to about 50% by volume of the X^{th} hypertonic solution from the peritoneal cavity and combining it with a second sugar concentrate to form an X^{th}+1 hypertonic solution;
(C) administering the X^{th}+1 hypertonic solution to the peritoneal cavity to form an X^{th}+2 hypertonic solution within the peritoneal cavity by mixing of the X^{th} and the X^{th}+1 hypertonic solutions;
(D) repeating steps (A) to (C) every 5 to 20 minutes for a treatment time of from about 1 to about 3 hours.
2. The method according to clause 1, wherein step (ii) comprises a tidal therapy phase and then a dwell phase.
3. The method according to clause 2, wherein step (ii) comprises a further tidal therapy phase following the dwell phase.
4. The method according to clause 1, wherein step (ii) comprises a dwell phase and then a tidal therapy phase.
5. The method according to clause 4, wherein step (ii) comprises a further dwell phase following the tidal therapy phase.
6. The method according to any one of the preceding clauses, wherein the first hypertonic solution has a sugar concentration of from about 1 wt % to about 4.5 wt %, for example about 1.5 wt % to about 4.25 wt %.
7. The method according to any one of the preceding clauses, wherein the sugar concentration of the N^{th} and N^{th}+1 hypertonic solutions within the peritoneal cavity during steps (a) to (d) is from about 0.8 wt % to about 4.5 wt %.
8. The method according to any one of the preceding clauses, wherein step (b) comprises passing the withdrawn N^{th} hypertonic solution through the dialysis sorbent followed by combining the withdrawn N^{th} hypertonic solution with the first sugar concentrate to form the N^{th}+1 hypertonic solution, wherein the first sugar concentrate comprises calcium ions and magnesium ions.
9. The method according to any one of clauses 1 to 7, wherein step (b) comprises combining the withdrawn N^{th} hypertonic solution with the first sugar concentrate to form the N^{th}+1 hypertonic solution, followed by:
   passing the N^{th}+1 hypertonic solution through the dialysis sorbent, and
   adding calcium and magnesium ions into the N^{th}+1 hypertonic solution after it has passed through the dialysis sorbent.
10. The method according to any one of the preceding clauses, wherein the calcium and magnesium ions are added into the N^{th}+1 hypertonic solution to give a calcium and magnesium ions concentration of from about 0.2 mM to about 3 mM, for example from about 0.25 mM to about 1.25 mM calcium and magnesium ions.
11. The method according to any one of the preceding clauses, wherein the calcium and magnesium ions concentration of the N^{th} and N^{th}+1 hypertonic solutions within the peritoneal cavity during steps (a) to (d) is from about 0.2 mM to about 3 mM, for example from about 0.25 mM to about 1.25 mM calcium and magnesium ions.
12. The method according to any one of the preceding clauses, wherein calcium, magnesium, and potassium ions are added into the N^{th}+1 hypertonic solution.
13. The method according to any one of the preceding clauses, wherein step (i) comprises administering from about 200 mL to about 4,000 mL, such as from about 400 mL to about 3,000 mL, such as from about 500 to about 2,500 mL of the first hypertonic solution to the peritoneal cavity.
14. The method according to any one of the preceding clauses, wherein step (i) comprises administering from about 1,000 mL to about 2,500 mL of the first hypertonic solution to the peritoneal cavity.
15. The method according to any one of the preceding clauses, wherein step (b) comprises withdrawing from about 100 mL to about 500 mL of the N^{th} hypertonic solution from the peritoneal cavity and combining the withdrawn N^{th} hypertonic solution with from about 0.1 mL to about 7 mL of the first sugar concentrate to form an N^{th}+1 hypertonic solution.
16. The method according to any one of the preceding clauses, wherein step (b) comprises withdrawing from about 200 mL to about 400 mL of the N^{th} hypertonic solution from the peritoneal cavity, for example from about 250 mL to about 300 mL.
17. The method according to any one of the preceding clauses, wherein step (b) comprises combining the withdrawn N^{th} hypertonic solution with from about 0.3 mL to about 6.8 mL of the first sugar concentrate, for example, from about 0.33 mL to about 3.0 mL, or from about 0.4 to about 2.8 mL.
18. The method according to clause 17, wherein step (b) comprises combining the withdrawn N^{th} hypertonic solution with from about 0.5 mL to about 2.4 mL, such as from about 0.6 mL to about 2.0 mL of the first sugar concentrate.
19. The method according to any one of the preceding clauses, wherein step (d) comprises repeating steps (a) to (c) every 7 to 17 minutes, for example, every 7.5 to 15 minutes, about every 7.5 minutes or about every 15 minutes.
20. The method according to any one of the preceding clauses, wherein the treatment time in step (d) is from 5 to 8 hours, for example 7 hours.
21. The method according to any one of the preceding clauses, wherein step (b) further comprises an initial step of saturating the dialysis sorbent with the first sugar concentrate.
22. The method according to any one of the preceding clauses, wherein the sugar concentration of the X^{th} and X^{th}+1 hypertonic solutions within the peritoneal cavity during steps (A) to (D) is from about 1.5 wt % to about 4.5 wt %.
23. The method according to any one of the preceding clauses, wherein step (B) comprises withdrawing from about 100 mL to about 500 mL of the X^{th} hypertonic solution from the peritoneal cavity and combining the withdrawn X^{th} hypertonic solution with from about 0.1 mL to about 7 mL of a second sugar concentrate comprising a sugar to form an X^{th}+1 hypertonic solution.
24. The method according to any one of the preceding clauses, wherein step (B) comprises withdrawing from about 200 mL to about 400 mL of the X^{th} hypertonic solution from the peritoneal cavity, for example from about 250 mL to about 300 mL.
25. The method according to any one of the preceding clauses, wherein step (B) comprises combining the withdrawn X^{th} hypertonic solution with from about 0.3 mL to about 6.8 mL of the second sugar concentrate, for example, from about 0.33 mL to about 3.0 mL, or from about 0.4 to about 2.8 mL.
26. The method according to clause 25, wherein step (B) comprises combining the withdrawn X^{th} hypertonic solution with from about 0.5 mL to about 2.4 mL, such as from about 0.6 mL to about 2.0 mL of the second sugar concentrate.
27. The method according to any one of the preceding clauses, wherein step (D) comprises repeating steps (A) to (C) every 5 to 10 minutes, for example, every 7.5 to 15 minutes, such as about every 7.5 minutes or about every 15 minutes.
28. The method according to any one of the preceding clauses, wherein the treatment time in step (D) is from 1.5 to 2.5 hours, for example 2 hours.
29. The method according to any one of the preceding clauses, wherein the first and second sugar concentrates each independently have a sugar concentration of from about 0.25 g/mL to about 0.9 g/mL, for example from about 0.65 g/mL to about 0.85 g/mL, such as about 0.7 g/mL.
30. The method according to any one of the preceding clauses, wherein the sugar in the first hypertonic solution, first sugar concentrate and/or second sugar concentrate is selected from one or more of the sugars selected from the groups consisting of glucose, icodextrin and sucrose, for example, the sugar may be glucose.
31. The method according to any one of the preceding clauses, wherein the calcium ions and/or magnesium ions present in the first sugar concentrate are derived from calcium lactate and/or magnesium lactate, respectively.
32. The method according to any one of the preceding clauses, wherein the second sugar concentrate is essentially free from metal ions, for example, essentially free from calcium and magnesium ions.
33. The method according to any one of the preceding clauses, wherein the second sugar concentrate consists essentially of a sugar and water.
34. The method according to any one of the preceding clauses, wherein the toxin in step (a) and/or (A) is a uremic toxin, for example urea.
35. The method according to any one of the preceding clauses, wherein steps (i) to (ii) achieve a Kt/V (urea) of at least about 1.7.

### DRAWINGS

FIG. 1 depicts dialysate to plasma (D/P) ratios for urea and creatinine during the standard peritoneal equilibration test (PET);

FIG. 2 depicts sorbent phase and non-sorbent phase therapy;

FIGS. 3A and 3B depict an apparatus suitable for performing the method disclosed herein in a no sorbent outflow dosing configuration and no sorbent inflow dosing configuration, respectively;

FIGS. 4A to 4C depict (a) 2×2-Way Valve with Glucose Dosing in Tidal Outflow, and (b) and (c) 2×2-Way Valve with Glucose Dosing in UF Only Outflow.

FIG. 5 depicts (a and b) the outflow phase and inflow phase of dose infusion with additional mixer.

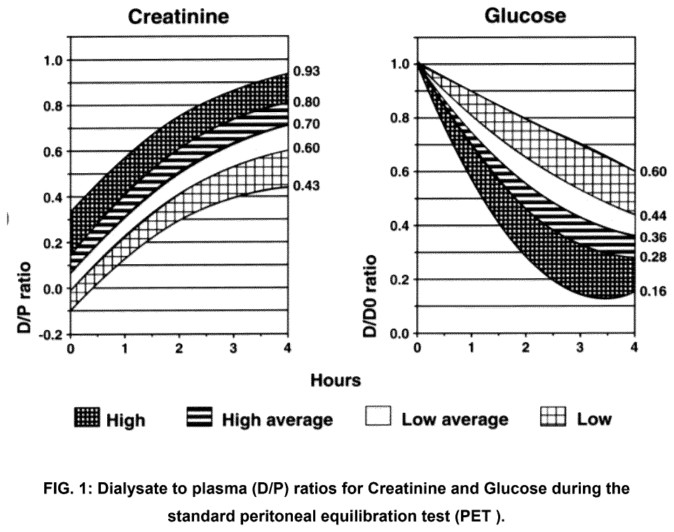

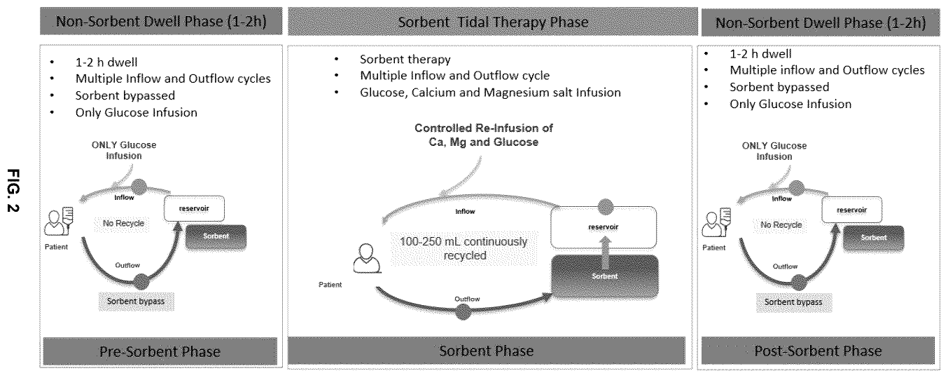

### DESCRIPTION

It has been surprisingly found that adding an additional non-sorbent phase before or after therapy allows one to achieve extra toxin clearance without the need for additional fluid and extra sorbent and it also facilitates additional water removal.

Thus, in an aspect of the invention, there is provided a method of peritoneal dialysis, the method comprising the steps of:
(i) administering to a subject a first hypertonic solution comprising a sugar to a peritoneal cavity in the subject, followed by;
(ii) a tidal therapy phase and then a dwell phase; or a dwell phase and then a tidal therapy phase;
wherein the tidal therapy phase comprises the steps of:
(a) allowing water and/or a toxin from the subject to pass into the peritoneal cavity by osmosis, thereby forming an N^{th} hypertonic solution within the peritoneal cavity;
(b) withdrawing up to about 50% by volume of the N^{th} hypertonic solution from the peritoneal cavity and combining it with a first sugar concentrate to form an N^{th}+1 hypertonic solution,
wherein the withdrawn N^{th} hypertonic solution and/or the N^{th}+1 hypertonic solution is passed through a dialysis sorbent, followed by adding calcium and magnesium ions into the withdrawn N^{th} hypertonic solution and/or the N^{th}+1 hypertonic solution;
(c) administering the N^{th}+1 hypertonic solution to the peritoneal cavity to form an N^{th}+2 hypertonic solution within the peritoneal cavity by mixing of the N^{th} and N^{th}+1 hypertonic solutions; and
(d) repeating steps (a) to (c) every 5 minutes to 30 minutes for a treatment time of about 5 hours to about 10 hours; and
wherein the dwell phase comprises the steps of:
(A) allowing water and/or a toxin from the subject to pass into the peritoneal cavity by osmosis, thereby forming an X^{th} hypertonic solution within the peritoneal cavity;
(B) withdrawing up to about 50% by volume of the X^{th} hypertonic solution from the peritoneal cavity and combining it with a second sugar concentrate to form an X^{th}+1 hypertonic solution;
(C) administering the X^{th}+1 hypertonic solution to the peritoneal cavity to form an X^{th}+2 hypertonic solution within the peritoneal cavity by mixing of the X^{th} and the X^{th}+1 hypertonic solutions;
(D) repeating steps (A) to (C) every 5 to 20 minutes for a treatment time of from about 1 to about 3 hours.

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

The phrase, “consists essentially of” and its pseudonyms may be interpreted herein to refer to a material where minor impurities may be present. For example, the material may be greater than or equal to 90% pure, such as greater than 95% pure, such as greater than 97% pure, such as greater than 99% pure, such as greater than 99.9% pure, such as greater than 99.99% pure, such as greater than 99.999% pure, such as 100% pure.

As used herein, the singular forms “a,” “an,” and “the” include plural referents unless the context clearly dictates otherwise. Thus, for example, reference to “a composition” includes mixtures of two or more such compositions, reference to “an oxygen carrier” includes mixtures of two or more such oxygen carriers, reference to “the catalyst” includes mixtures of two or more such catalysts, and the like.

The terms “subject” and “subjects” include references to mammalian (e.g. human) subjects. As used herein the terms “subject” or “patient” are well-recognized in the art, and, are used interchangeably herein to refer to a mammal, including dog, cat, rat, mouse, monkey, cow, horse, goat, sheep, pig, camel, and, most preferably, a human. In some embodiments, the subject is a subject in need of treatment or a subject with a disease or disorder. However, in other embodiments, the subject can be a normal subject. The term does not denote a particular age or sex. Thus, adult and newborn subjects, whether male or female, are intended to be covered. In certain embodiments of the invention, the subject is an adult human.

The term “about” as used herein can allow for a degree of variability in a value or range, for example, within 10%, within 5%, within 1%, within 0.5%, within 0.1%, within 0.05%, within 0.01%, within 0.005%, or within 0.001% of a stated value or of a stated limit of a range, and includes the exact stated value or range.

The term “% wt” as used herein refers to the percentage mass of a particular substance in a solution with respect to the total mass of the solution, unless indicated otherwise. For example, a hypertonic solution disclosed herein containing 1.5 wt % of a sugar is to be understood as containing 1.5 g of the sugar in every 100 g of the hypertonic solution.

As used herein, the terms “toxin” or “toxins” refers to organic compounds which accumulate in the bloodstream and cannot be eliminated from the body. The terms encompass uremic toxins and protein bound uremic toxins (PBUT). The term “uremic toxins” includes, but is not limited to, urea, uric acid, creatinine, p-cresyl sulfate, indoxyl sulfate, beta-2-microglubulin, and inorganic phosphate.

In certain embodiments of the invention, the toxin in step (a) and/or (A) may be a uremic toxin, for example urea.

When used herein, the term “tolerable maximum volume” can be determined by each subject who undergoes the method of treatment. A tolerable maximum volume for a given subject may be determined by a skilled physician. Examples of the maximum tolerable volume for a human may include volumes up to, and exceeding 4,000 mL. Examples of suitable volumes that may be used include, but are not limited to from 300 mL to 4,000 mL, such as from 400 mL to 3,000 mL such as from 500 mL to 2,500 mL of a first hypertonic solution comprising a sugar to the peritoneal cavity.

Any suitable sugar concentration may be used in the hypertonic solutions.

In certain embodiments of the invention, the first hypertonic solution may have a sugar concentration of from about 1 wt % to about 4.5 wt %, for example about 1.5 wt % to about 4.25 wt %.

In certain embodiments of the invention, the sugar concentration of the N^{th} and N^{th}+1 hypertonic solutions within the peritoneal cavity during steps (a) to (d) of the present method may be from about 0.8 wt % to about 4.5 wt %

Any suitable calcium and magnesium ions concentration may be used.

In certain embodiments of the invention, the calcium and magnesium ions may be added into the N^{th}+1 hypertonic solution to give a calcium and magnesium ions concentration of from about 0.2 mM to about 3 mM, for example from about 0.25 mM to about 1.25 mM calcium and magnesium ions. It will be appreciated that the concentration refers to the combined concentration of calcium and magnesium ions, rather than the individual concentrations of the calcium and magnesium ions.

In certain embodiments of the invention, the calcium and magnesium ions concentration of the N^{th} and N^{th}+1 hypertonic solutions within the peritoneal cavity during steps (a) to (d) of the present method may be from about 0.2 mM to about 3 mM, for example from about 0.25 mM to about 1.25 mM calcium and magnesium ions.

Any suitable calcium and magnesium salts may be used to provide calcium and magnesium ions, respectively. Examples of suitable calcium salts include, but are not limited to, calcium lactate and calcium chloride. Examples of suitable magnesium salts include, but are not limited to, magnesium lactate and magnesium chloride. In certain embodiments of the invention, the calcium ions and/or magnesium ions present in the first sugar concentrate may be derived from calcium lactate and/or magnesium lactate, respectively.

Any suitable potassium salt may be used to provide potassium ions. For example, potassium ions may be derived from potassium chloride. Any suitable concentration of potassium ions may be used. For example, the concentration of potassium ions may range between 0.5 mM to 5 mM.

For the avoidance of doubt, it is explicitly contemplated that where a number of numerical ranges related to the same feature are cited herein, that the end points for each range are intended to be combined in any order to provide further contemplated (and implicitly disclosed) ranges. Thus, in step (i) of the method, the following volume ranges of the first hypertonic solution are expressly contemplated:
from 200 to 300 mL, from 200 to 400 mL, from 200 to 500 mL, from 200 to 1,000 mL, from 200 to 2,500 mL, from 200 to 3,000 mL, from 200 to 4,000 mL, from 200 mL to a tolerable maximum volume for the subject;
from 300 to 400 mL, from 300 to 500 mL, from 300 to 1,000 mL, from 300 to 2,500 mL, from 300 to 3,000 mL, from 300 to 4,000 mL, from 300 mL to a tolerable maximum volume for the subject;
from 400 to 500 mL, from 400 to 1,000 mL, from 400 to 2,500 mL, from 400 to 3,000 mL, from 400 to 4,000 mL, from 400 mL to a tolerable maximum volume for the subject;
from 500 to 1,000 mL, from 500 to 2,500 mL, from 500 to 3,000 mL, from 500 to 4,000 mL, from 500 mL to a tolerable maximum volume for the subject;
from 1,000 to 2,500 mL, from 1,000 to 3,000 mL, from 1,000 to 4,000 mL, from 1,000 mL to a tolerable maximum volume for the subject;
from 2,500 to 3,000 mL, from 2,500 to 4,000 mL, from 2,500 mL to a tolerable maximum volume for the subject;
from 3,000 to 4,000 mL, from 3,000 mL to a tolerable maximum volume for the subject; and from 4,000 mL to a tolerable maximum volume for the subject.

The above analysis is applicable to all other sets of numerical ranges disclosed herein.

In certain embodiments of the invention, step (ii) of the present method may comprise a tidal therapy phase and then a dwell phase. In such embodiments, step (ii) of the present method may comprise a further tidal therapy phase following the dwell phase.

In certain embodiments of the invention, step (ii) of the present method may comprise a dwell phase and then a tidal therapy phase. In such embodiments, step (ii) of the present method may comprise a further dwell phase following the tidal therapy phase.

As will be appreciated, the method disclosed herein is a tidal system. As such, the steps of the method disclosed herein are repeated in a cyclical manner over a suitable period of time to have the desired effect.

In certain embodiments of the invention, step (b) of the present method may comprise passing the withdrawn N^{th} hypertonic solution through the dialysis sorbent followed by combining the withdrawn N^{th} hypertonic solution with the first sugar concentrate to form the N^{th}+1 hypertonic solution, wherein the first sugar concentrate comprises calcium ions and magnesium ions.

In certain embodiments of the invention, step (b) of the present method may comprise combining the withdrawn N^{th} hypertonic solution with the first sugar concentrate to form the N^{th}+1 hypertonic solution, followed by:
passing the N^{th}+1 hypertonic solution through the dialysis sorbent, and
adding calcium and magnesium ions into the N^{th}+1 hypertonic solution after it has passed through the dialysis sorbent.

In certain embodiments of the invention, step (b) of the present method may comprise withdrawing up to about 50% by volume of the N^{th} hypertonic solution from the peritoneal cavity, for example, about 1% to about 50%, 5% to 40%, 10% to 40%, 10% to 30% or 10% to 20%.

In certain embodiments of the invention, step (b) of the present method may comprise withdrawing from about 100 mL to about 500 mL of the N^{th} hypertonic solution from the peritoneal cavity and combining the withdrawn N^{th} hypertonic solution with from about 0.1 mL to about 7 mL of the first sugar concentrate to form an N^{th}+1 hypertonic solution.

In certain embodiments of the invention, step (b) of the present method may comprise withdrawing from about 200 mL to about 400 mL of the N^{th} hypertonic solution from the peritoneal cavity, for example from about 250 mL to about 300 mL.

In certain embodiments of the invention, step (b) of the present method may comprise combining the withdrawn N^{th} hypertonic solution with from about 0.3 mL to about 6.8 mL of the first sugar concentrate, for example, from about 0.33 mL to about 3.0 mL, or from about 0.4 to about 2.8 mL.

In certain embodiments of the invention, step (b) of the present method may comprise combining the withdrawn N^{th} hypertonic solution with from about 0.5 mL to about 2.4 mL, such as from about 0.6 mL to about 2.0 mL of the first sugar concentrate.

In certain embodiments of the invention, step (b) of the present method may further comprise an initial step of saturating the dialysis sorbent with the first sugar concentrate. The dialysis sorbent may be saturated with the first sugar concentrate by any suitable method. For example, the dialysis sorbent may be saturated with the first sugar concentrate by an initial fill used for a patient. The initial fill may be 1.5% Dianeal®, 2.5% Dianeal® or 4.25% Dianeal®, which is typically used as the initial fill for a patient. The volume of the fluid used to saturate the sorbent may vary between 100 mL to 1000 mL.

For example, steps (a) to (c) of the method may be repeated every 5 to 30 minutes (e.g. every 10 to 20 minutes) for a desired treatment time. For example, steps (a) to (c) may be repeated every 7 to 17 minutes, such as every 7.5 to 15 minutes, such as about every 7.5 minutes or about every 15 minutes for a desired treatment time (for example, a treatment time of about 5 to 10 hours, such as about 7 hours). And, for example, steps (A) to (C) of the method may be repeated every 5 to 20 minutes for a desired treatment time. For example, steps (A) to (C) may be repeated every 7 to 17 minutes, such as every 7.5 to 15 minutes, such as about every 7.5 minutes or about every 15 minutes for a desired treatment time (for example, a treatment time of about 1 to 3 hours, such as about 2 hours).

Any suitable period of time determined by the skilled physician may be used herein. Examples of suitable desired treatment times for step (v) may be from 7 to 10 hours, though longer (or shorter) times may be selected by the physician based upon their own knowledge of the subject in question. Examples of suitable desired treatment times for step (xi) may be from 1 to 3 hours, though longer (or shorter) times may be selected by the physician based upon their own knowledge of the subject in question. Examples of suitable desired treatment times for step (d) may be from 5 to 8 hours (e.g. 7 hours), though longer (or shorter) times may be selected by the physician based upon their own knowledge of the subject in question.

Examples of suitable desired treatment times for step (D) may be from 1.5 to 2.5 hours (e.g. 2 hours), though longer (or shorter) times may be selected by the physician based upon their own knowledge of the subject in question.

In certain embodiments of the invention, the dwell phase(s) of the present method does not comprise a step of passing a hypertonic solution through a dialysis sorbent.

As used herein, “N” is an integer, for example, from 1 to 100, such as 1 to 60. The N^{th} hypertonic solutions are formed during the tidal therapy phase comprising the steps (a) to (d) of the method. For example, in the method disclosed herein hypertonic solution formed in step (a) may be a 1^{st} hypertonic solution of the tidal therapy phase, the hypertonic solution formed in step (b) may be a 2^{nd} (i.e. N^{th}+1) hypertonic solution of the tidal therapy phase, and the hypertonic solution formed in step (c) may be a 3^{rd} (N^{th}+2) hypertonic solution of the tidal therapy phase.

In addition, as used herein, “X” is an integer, for example, from 1 to 100, such as 1 to 60. The X^{th} hypertonic solutions are formed during the dwell phase comprising the steps (A) to (D) of the method. For example, in the method disclosed herein the hypertonic solution formed in step (A) may be a 1^{st} hypertonic solution (i.e. X^{th}) of the dwell phase, the hypertonic solution formed in step (C) may be a 2^{nd} (i.e. X^{th}+1) hypertonic solution of the dwell phase, and the hypertonic solution formed in step (C) may be a 3^{rd} (X^{th}+2) hypertonic solution of the dwell phase.

The standard dwell phase Kt/V urea clearance can be determined by calculating Xurea based on Equation 1.

$${X}_{u r e a} = \frac{F {D}_{urea}}{{B}_{urea}} \times \frac{{V}_{F D}}{{V}_{T B W}} \qquad Equation 1$$

FDᵤᵣₑₐ=Urea concentration (mmol/L) in the final volume
Bᵤᵣₑₐ=Urea concentration (mmol/L) in blood
V_{FD}=Final volume (L)
V_{TBW}=Volume of total body water (L)
The parameters Bᵤᵣₑₐ, B_{crea}, B_{b2m}, and V_{TBW} are predetermined.

Due to the difference in toxin concentrations, the Equation 1 for the final volume above cannot be used to measure the toxin concentrations in the tidal volumes. Samples of the tidal volume should be taken during therapy to better represent the toxins that are absorbed by the sorbent material. Additional parameters are used to measure the toxin clearance based on samples of the tidal volumes of the waste dialysate during therapy. The standard Kt/V urea clearance can be determined by calculating Yᵤᵣₑₐ based on Equation 2.

$${Y}_{u r e a} = \frac{T {D}_{urea}}{{B}_{urea}} \times \frac{{V}_{T D}}{{V}_{T B W}} \times N \qquad Equation 2$$

TDᵤᵣₑₐ=Urea concentration (mmol/L) in the tidal volume
V_{TD}=Tidal volume (L)
N=Device efficiency factor (%)
The parameters V_{TD} and N are predetermined.

Total kt/V of the therapy is obtained by sum of Kt/V for the sorbent phase and non-sorbent phase

$$Kt / V ( therapy ) = {X}_{u r e a} + {Y}_{u r e a}$$

In certain embodiments of the invention, steps (i) to (ii) of the present method may achieve a Kt/V(urea) of at least about 1.7.

In certain embodiments of the invention, the sugar concentration of the X^{th} and X^{th}+1 hypertonic solutions within the peritoneal cavity during steps (A) to (D) may be from about 1.5 wt % to about 4.5 wt %.

In certain embodiments of the invention, step (B) of the present method may comprise withdrawing up to about 50% by volume of the X^{th} hypertonic solution from the peritoneal cavity, for example, about 1% to about 50%, 5% to 40%, 10% to 40%, 10% to 30% or 10% to 20%.

In certain embodiments of the invention, step (B) of the present method may comprise withdrawing from about 100 mL to about 500 mL of the X^{th} hypertonic solution from the peritoneal cavity and combining the withdrawn X^{th} hypertonic solution with from about 0.1 mL to about 7 mL of a second sugar concentrate comprising a sugar to form an X^{th}+1 hypertonic solution.

In certain embodiments of the invention, step (B) of the present method may comprise withdrawing from about 200 mL to about 400 mL of the X^{th} hypertonic solution from the peritoneal cavity, for example from about 250 mL to about 300 mL.

In certain embodiments of the invention, step (B) of the present method may comprise combining the withdrawn X^{th} hypertonic solution with from about 0.3 mL to about 6.8 mL of the second sugar concentrate, for example, from about 0.33 mL to about 3.0 mL, or from about 0.4 to about 2.8 mL.

In certain embodiments of the invention, step (B) of the present method may comprise combining the withdrawn X^{th} hypertonic solution with from about 0.5 mL to about 2.4 mL, such as from about 0.6 mL to about 2.0 mL of the second sugar concentrate.

As noted herein, the method makes use of a sugar concentrate. This sugar concentrate may contain any suitable sugar. For example, the sugar may be selected from glucose, sucrose, or icodextrin. In particular embodiments of the invention, the sugar concentrate may comprise (or consist of) glucose and water. As will be appreciated, the same sugar may be used in the initial hypertonic solution.

In certain embodiments of the invention, the second sugar concentrate may be essentially free from metal ions, for example, essentially free from calcium and magnesium ions. For example, the second sugar concentrate may consist essentially of a sugar and water.

The maximum total dosage of the sugar based osmotic agent per treatment may be based on the current gold-standard of care, which is 14 L of an aqueous 2.5 wt % glucose solution. It will be appreciated that the methods disclosed herein enables the amount of glucose (or other sugar) to be reduced significantly compared to this maximum value.

In embodiments of the invention that may be mentioned herein, the sugar concentration of the withdrawn N^{th} hypertonic solution in each repetition of step (a) varies by less than 50%, such as less than 40%, relative to the initial sugar-based osmotic agent concentration of the withdrawn N^{th} hypertonic solution the first time step (a) is performed.

In certain embodiments of the invention, the N^{th}+1 hypertonic solution may have a higher sugar concentration than the initial hypertonic solution.

In certain embodiments of the invention, the N^{th}+2 hypertonic solution may have a sugar concentration that is less than or equal to the sugar concentration of the initial hypertonic solution.

In certain embodiments of the invention, the first and second sugar concentrates may each independently have a sugar concentration of from about 0.25 g/mL to about 0.9 g/mL, for example from about 0.65 g/mL to about 0.85 g/mL, such as about 0.7 g/mL.

An apparatus suitable for performing the method disclosed herein is the AWAK Advanced Glucose Management System (AWAK AGMS). This system is designed to allow a physician to adjust the amount of glucose that is dosed during each tidal cycle in order to regulate and target the required ultrafiltration removal.

Thus, an example of an apparatus suitable for performing the method disclosed herein comprises:
a first pump fluidly connectable to a subject's peritoneum;
a sugar concentrate supply pump connectable to a source of sugar concentrate;
a storage chamber; and
a first fluid flow path from the first pump to the storage chamber;
where the first pump is configured to pump fluid in either direction along the first fluid flow paths,
such that when in use:
   fluid can be drawn from a subject's peritoneum along the first fluid flow path by the first pump to the storage chamber and
   the sugar concentrate supply pump is configured to supply a sugar concentrate to fluid flowing along the first fluid flow path.

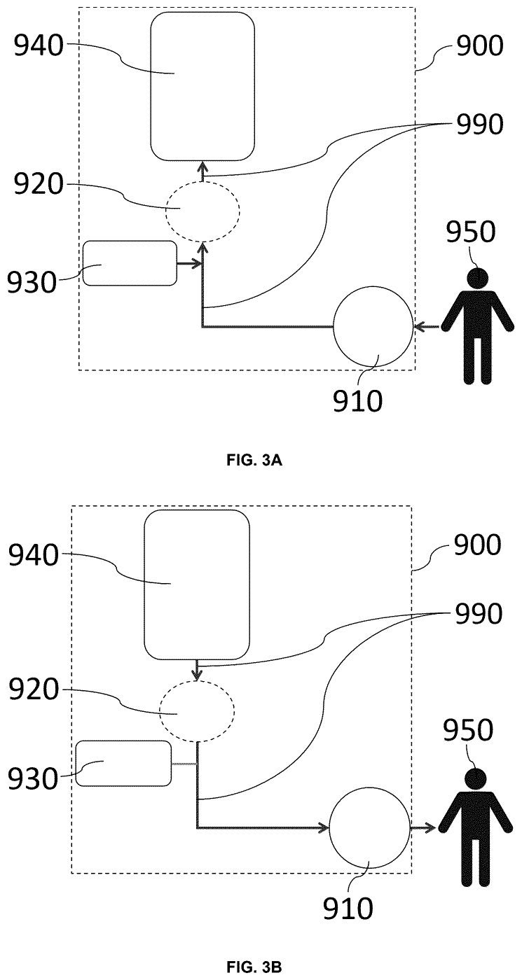

The above mentioned apparatus will be briefly discussed by reference to FIG. 3. As noted above the apparatus 900 comprises
a first pump 910 fluidly connectable to a subject's 950 peritoneum;
a sugar concentrate supply pump 930 connectable to a source of sugar concentrate;
a storage chamber 940; and
a first fluid flow path 990 from the first pump 910 to the storage chamber 940;
where the first pump 910 is configured to pump fluid in either direction along the first fluid flow path,
such that when in use:
   fluid can be drawn from a subject's peritoneum along the first fluid flow path by the first pump to the storage chamber; and
   the sugar concentrate supply pump is configured to supply a sugar concentrate to fluid flowing along the first fluid flow path.

While not shown, the apparatus of FIG. 3 may be connected to a controller that configured to operate the apparatus. It will be appreciated that any suitable controller may be used. For example, the controller may be an Arduino Due and relevant supporting electronics, which may include pressure sensors etc.

It will be appreciated that the sugar concentrate can be added either in an outflow sense (i.e. when fluid is being drawn from a subject), in an inflow sense (i.e. when fluid is being returned to a subject) or in both senses, depending on the need of the subject and the physician's instructions.

It will be appreciated that there will be a certain amount of turbulence associated with the flow of the fluid resulting in mixing of the sugar concentrate with the bulk of the fluid through the movement of the fluid through the system, storage in the storage tank or while in the peritoneal cavity of a subject. As such there may be no need to include any mixing means or apparatus as part of the apparatus. However, in some embodiments of the invention, a mixing means or apparatus, such as a mixer 920 may be placed within the first fluid flow path. As will be appreciated, the exact location of the mixer(s) will depend on how the apparatus is intended to function. For example, the mixer 920 may be a single mixer that is placed downstream relative to the direction of flow used to introduce the sugar concentrate. As shown in FIG. 3A, the mixer 920 is placed between the sugar concentrate supply pump 930 and the storage chamber 940. In other embodiments, the mixer 920 may be placed so that it is upstream of the sugar concentrate supply pump 930 in an inflow sense, such that the sugar concentrate supply pump 930 and the storage chamber 940 are not separated by the mixer. This allows for the introduction and mixing of the sugar concentrate during an inflow phase of the apparatus. Alternatively, there may be more than one mixer in the first fluid flow path, allowing mixing to take place both in an inflow and outflow phase of the apparatus. It will be understood that the placement of mixers discussed here is general and applies to all other embodiments discussed herein.

As will be appreciated, the apparatus disclosed herein may be used in conjunction with a control means or apparatus. This control means or apparatus is configured to operate the apparatus and may be, in particular embodiments mentioned herein, configured to implement the methods described herein below. As will be appreciated, the control means or apparatus may be a reusable component that can be connected to and then removed from a disposable apparatus.

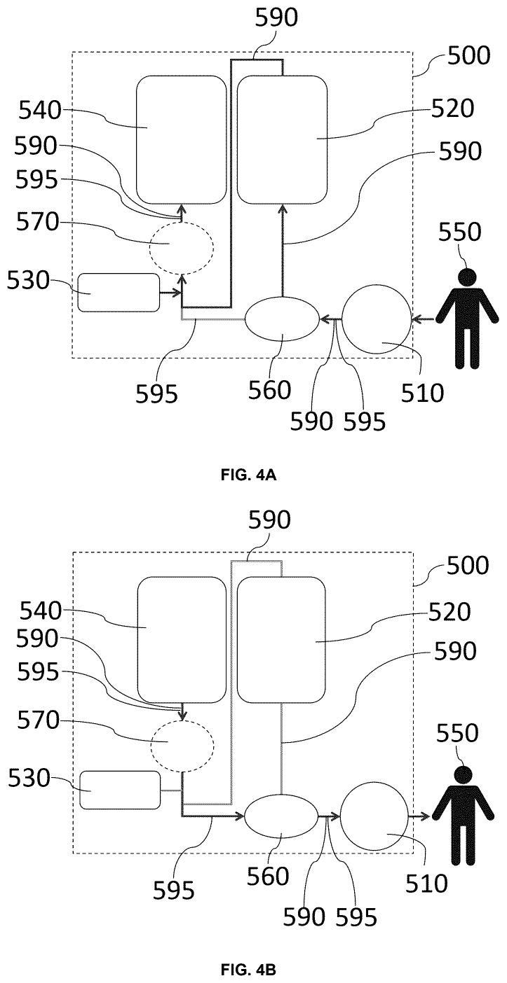

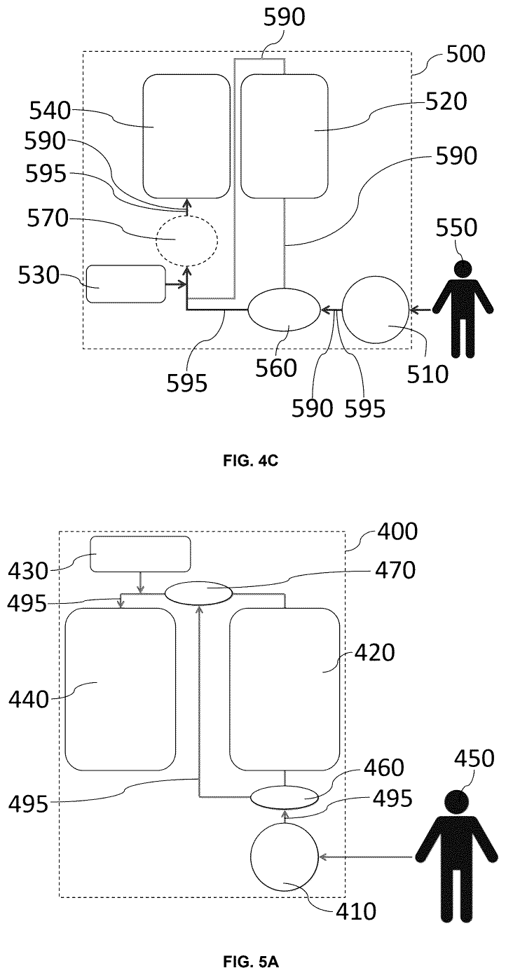

The above apparatus may be suitable for the removal of fluid from a subject only, without necessarily dealing with the removal of a substantial amount of toxins from the subject. With that in mind, for the first cycle of the method disclosed herein the apparatus includes a sorbent that enables toxins to be removed, thereby allowing peritoneal dialysis to take place at the same time as removing fluid from the subject. Such an apparatus will be briefly discussed by reference to FIG. 4. As noted above the apparatus 500 comprises
a first pump 510 fluidly connectable to a subject's 550 peritoneum;
a sugar concentrate supply pump 530 connectable to a source of sugar concentrate;
a storage chamber 540;
a first fluid flow path 590 from the first pump 510 to the storage chamber 540;
a dialysis sorbent 520 situated in the first fluid flow path; and
a second fluid flow 595 path from the first pump 510 to the storage chamber 540 that bypasses the sorbent 512;
where the first pump 510 is configured to pump fluid in either direction along the first and second fluid flow paths,
such that when in use:
   fluid can be drawn from a subject's peritoneum along the first fluid or second flow path by the first pump to the storage chamber; and
   the sugar concentrate supply pump is configured to supply a sugar concentrate to fluid flowing along the first fluid or second flow paths.

While not shown, the apparatus of FIG. 4 may be connected to a controller that configured to operate the apparatus, where the controller can select whether to use the first or the second fluid flow path for any fluid flow operation.

The dialysis sorbent mentioned herein may be any suitable dialysis sorbent and is not particularly limited. The only requirement is that it can be packed into a suitable chamber within the apparatus. Examples of sorbents include, but are not limited to those described in PCT Application No. PCT/SG2009/000229, which is hereby incorporated by reference.

As will be appreciated, the dialysis sorbent will be housed in a suitable chamber within the apparatus disclosed herein. If the apparatus is disposable, the sorbent may be housed within the chamber directly. However, if the apparatus is intended to be partly re-usable, the sorbent may be stored within a separate sorbent cartridge that may be placed into the apparatus before use. The former arrangement, where the sorbent is directly held within a chamber, thereby allowing the apparatus to be disposable in nature after a single use, which may be beneficial for hygiene reasons.

As will be appreciated, the pumps may be part of a permanent apparatus section and are not disposable. They may be connected to the controller for this purpose. The apparatus may have a disposable section which consists of the tubing, flow paths, dialysis sorbent (when present) and storage chamber, plus any connections to, for example, an ultrafiltration bag.

The apparatus of FIG. 4 allows one to select at each outflow phase whether to pass the fluid from a subject through the sorbent or not. This may be achieved by the use of any suitable means or apparatus that allows for such control. For example the apparatus may make use of one or more values configured to selectively enable fluid flow through one of the first and second fluid flow paths. The embodiment of FIG. 4 may include a three-way valve 560 that enables the selection of the desired flow path under the influence of the controller.

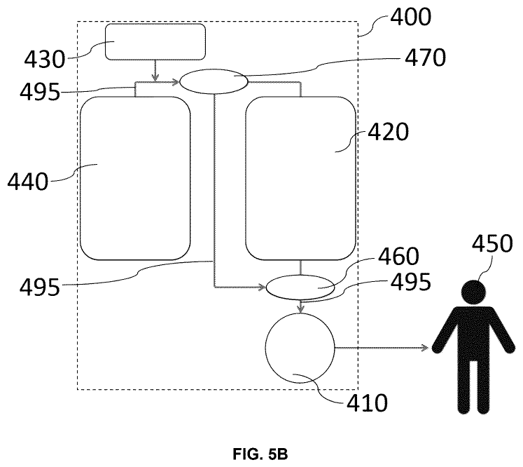

An alternative embodiment of the apparatus may make use of two or more valves configured to selectively enable fluid flow through one of the first and second fluid flow paths. For example, the embodiment depicted in FIG. 5 makes use of two values 460 and 470, which allows for greater control. That is, the use of two valves in the configuration depicted allows for the sorbent to be used either during the inflow phase or the outflow phase, as desired. In other words, in embodiments of the invention that may be mentioned herein, the dialysis sorbent may be situated upstream or downstream of the storage chamber in the first fluid flow path. For completes, in FIG. 5, there is an apparatus 400 and a subject 450. The apparatus 400 includes a first pump 410, a dialysis sorbent 420, a sugar concentrate supply pump 430, a storage chamber 440, a first valve 460, a second valve 470, a first fluid flow path (not shown) and a second fluid flow path 495. The sugar concentrate supply pump 430 is supplied to the fluid at a location between the storage chamber 440 and the second valve 470.

As will be appreciated, one or more mixers may be located in the first and second flow paths (e.g. 570 in FIG. 4) and these may be configured in a similar manner to that described above.

In embodiments that may be mentioned herein, the sugar concentrate supply pump may be configured to supply sugar concentrate to fluid in the second fluid flow path. This is shown in FIG. 4. For the avoidance of doubt, the sugar concentrate supply pump may be configured to supply sugar concentrate to fluid in the first fluid flow path should that be desired.

Further aspects and embodiments of the invention will now be described by reference to the following non-limiting examples.

### EXAMPLES

### Materials

| | Chemicals | CAS Number |
|---|---|---|
| 1 | Dextrose monohydrate, USP | 14431-43-7 |
| 2 | Sodium Chloride, USP | 7647-14-5 |
| 3 | Sodium Lactate | 72-17-3 |
| 4 | Calcium Chloride dihydrate, USP | 10035-04-8 |
| 5 | Magnesium chloride | 7791-18-6 |
| | hexahydrate, USP | |

### Modelling Technique

A proprietary modelling software was used to estimate in silco the efficacy (Kt/V) of various treatments methods for human patient of different transport status and different weight group. The modelling software was based on a mathematical model that describes the flow of solutes and water across the peritoneal membrane during a peritoneal dialysis therapy.

In the modelling, the hypertonic solution for the initial fill (i.e. step (i) of the claimed method) was set as being 1.5% or 2.5% Low calcium Dianeal®.

Composition/100 mL

| | Dextrose, | Sodium | Sodium | Calcium | Magnesium |
|---|---|---|---|---|---|
| | Hydrous, | Chloride, | Lactate | Chloride, USP | Chloride, USP |
| | USP | USP (NaCl) | (C₃H₅NaO₃) | (CaCl₂•2H₂O) | (MgCl₂•H₂O) |
| Dianeal ® | 1.5 g | 538 mg | 448 mg | 18.3 mg | 5.08 mg |
| Low | | | | | |
| Calcium | | | | | |
| Peritoneal | | | | | |
| Dialysis | | | | | |
| Solution | | | | | |
| with 1.5% | | | | | |
| Dextrose | | | | | |
| Dianeal ® | 2.5 g | 538 mg | 448 mg | 18.3 mg | 5.08 mg |
| Low | | | | | |
| Calcium | | | | | |
| Peritoneal | | | | | |
| Dialysis | | | | | |
| Solution | | | | | |
| with 2.5% | | | | | |
| Dextrose | | | | | |
| Dianeal ® | 4.25 g | 538 mg | 448 mg | 18.3 mg | 5.08 mg |
| Low | | | | | |
| Calcium | | | | | |
| Peritoneal | | | | | |
| Dialysis | | | | | |
| Solution | | | | | |
| with | | | | | |
| 4.25% | | | | | |
| Dextrose | | | | | |

### Example 1

### Kt/V(urea) for Patient With 2L of 1.5% Low Calcium Dianeal® as Initial Fill and 7-Hour Tidal Peritoneal Dialysis as Method of Treatment

| | Patient Size | | | | |
|---|---|---|---|---|---|
| | 25 L | 30 L | 40 L | 50 L | 60 L |
| Transport Type | (45 kg) | (55 kg) | (73 kg) | (91 kg) | (109 kg) |
| High | 1.58 | 1.34 | 1.03 | 0.83 | 0.70 |
| High-Average | 1.51 | 1.28 | 0.98 | 0.79 | 0.66 |
| Low-Average | 1.37 | 1.16 | 0.88 | 0.71 | 0.60 |
| Low | 1.32 | 1.12 | 0.85 | 0.69 | 0.58 |

### Example 2

### Kt/V(urea) for Patient With 2L of 1.5% Low Calcium Dianeal® as Initial Fill and 10-Hour Tidal Peritoneal Dialysis as Method of Treatment

| | Patient Size | | | | |
|---|---|---|---|---|---|
| | 25 L | 30 L | 40 L | 50 L | 60 L |
| Transport Type | (45 kg) | (55 kg) | 73 kg) | 91 kg) | (109 kg) |
| High | 2.10 | 1.78 | 1.37 | 1.11 | 0.93 |
| High-Average | 2.02 | 1.71 | 1.31 | 1.06 | 0.89 |
| Low-Average | 1.85 | 1.56 | 1.19 | 0.97 | 0.81 |
| Low | 1.79 | 1.51 | 1.15 | 0.93 | 0.78 |

### Example 3

### Kt/V(urea) for Patient With 2L of 1.5% Low Calcium Dianeal® as Initial Fill and 1 Hour Pre-Dwell+7-Hour Tidal Peritoneal Dialysis as Method of Treatment

| | Patient Size | | | | |
|---|---|---|---|---|---|
| | 25 L | 30 L | 40 L | 50 L | 60 L |
| Transport Type | (45 kg) | (55 kg) | (73 kg) | (91 kg) | (109 kg) |
| High | 1.68 | 1.42 | 1.09 | 0.88 | 0.74 |
| High-Average | 1.62 | 1.37 | 1.05 | 0.85 | 0.71 |
| Low-Average | 1.48 | 1.25 | 0.95 | 0.77 | 0.65 |
| Low | 1.43 | 1.21 | 0.92 | 0.75 | 0.63 |

### Example 4

### Kt/V(urea) for Patient With 2L of 1.5% Low Calcium Dianeal® as Initial Fill and 2-Hour Pre-Dwell+7-Hour Tidal Peritoneal Dialysis as Method of Treatment

| | Patient Size | | | | |
|---|---|---|---|---|---|
| | 25 L | 30 L | 40 L | 50 L | 60 L |
| Transport Type | (45 kg) | (55 kg) | (73 kg) | (91 kg) | (109 kg) |
| High | 1.73 | 1.47 | 1.12 | 0.91 | 0.76 |
| High-Average | 1.68 | 1.42 | 1.09 | 0.88 | 0.74 |
| Low-Average | 1.55 | 1.31 | 1.00 | 0.81 | 0.68 |
| Low | 1.51 | 1.28 | 0.97 | 0.79 | 0.66 |

### Example 5

### Kt/V(urea) for Patient With 2L of 1.5% Low Calcium Dianeal® as Initial Fill and 7 Hour Tidal Peritoneal Dialysis+1 Hour Post Sorbent Tidal Dialysis Dwell as Method of Treatment

| | Patient Size | | | | |
|---|---|---|---|---|---|
| | 25 L | 30 L | 40 L | 50 L | 60 L |
| Transport Type | (45 kg) | (55 kg) | (73 kg) | (91 kg) | (109 kg) |
| High | 1.69 | 1.43 | 1.10 | 0.89 | 0.75 |
| High-Average | 1.63 | 1.38 | 1.06 | 0.86 | 0.72 |
| Low-Average | 1.49 | 1.26 | 0.96 | 0.78 | 0.65 |
| Low | 1.45 | 1.22 | 0.93 | 0.75 | 0.63 |

### Example 6

### Kt/V(urea) for Patient With 2L of 1.5% Low Calcium Dianeal® as Initial Fill and 7-Hour Tidal Peritoneal Dialysis+2-Hour Post Sorbent Tidal Dialysis Dwell as Method of Treatment

| | Patient Size | | | | |
|---|---|---|---|---|---|
| | 25 L | 30 L | 40 L | 50 L | 60 L |
| Transport Type | (45 kg) | (55 kg) | (73 kg) | (91 kg) | (109 kg) |
| High | 1.75 | 1.48 | 1.14 | 0.92 | 0.78 |
| High-Average | 1.71 | 1.45 | 1.11 | 0.90 | 0.76 |
| Low-Average | 1.58 | 1.34 | 1.02 | 0.83 | 0.69 |
| Low | 1.55 | 1.31 | 1.00 | 0.81 | 0.68 |

### Example 7

### Kt/V(urea) for Patient With 2L of 1.5% Low Calcium Dianeal® as Initial Fill and 1 Hour Pre-Dwell+7-Hour Tidal Peritoneal Dialysis+2-Hour Post Sorbent Tidal Dialysis Dwell as Method of Treatment

| | Patient Size | | | | |
|---|---|---|---|---|---|
| | 25 L | 30 L | 40 L | 50 L | 60 L |
| Transport Type | (45 kg) | (55 kg) | (73 kg) | (91 kg) | (109 kg) |
| High | 1.85 | 1.57 | 1.20 | 0.97 | 0.82 |
| High-Average | 1.82 | 1.54 | 1.18 | 0.96 | 0.80 |
| Low-Average | 1.69 | 1.43 | 1.09 | 0.88 | 0.74 |
| Low | 1.66 | 1.40 | 1.07 | 0.87 | 0.73 |

### Example 8

### Comparison of Ultrafiltration (L) Using 2L of 1.5% Low Calcium Dianeal® as Initial Fill for 7-Hour Tidal Therapy and 7 Hour Tidal+2 Hour Post Tidal Therapy Under Low and High Glucose Infusion

A low glucose setting means an infusion of 70% glucose at a rate of 0.4 ml/cycle and a high glucose setting means an infusion of 70% glucose at a rate of 1.2 mL/cycle. A medium glucose setting may refer to an infusion of 70% glucose solution at a rate of 0.8 mL/cycle.

| | Low Glucose Setting | | High Glucose Setting | |
|---|---|---|---|---|
| | | Tidal-7 hour + | | Tidal-7 hour + |
| Transport | UF volume (L) | 2-hour Post | UF volume (L) | 2-hour Post |
| status | Tidal-7 hour | sorbent | Tidal-7 hour | sorbent |
| High | − 0.2 | − 0.3 | 0.1 | 0.1 |
| High-Average | 0.0 | 0.0 | 0.4 | 0.5 |
| Low-Average | 0.1 | 0.1 | 0.4 | 0.6 |
| Low | 0.3 | 0.4 | 0.7 | 1.0 |

### Example 9

### Comparison of Predicted Ultrafiltration (L) Using 2L of 2.5% Low Calcium Dianeal® as Initial Fill for 7-Hour Tidal Therapy and 7 Hour Tidal+2 Hour Post Tidal Therapy Under Low and High Glucose Infusion

| | Low Glucose Setting | | High Glucose Setting | |
|---|---|---|---|---|
| | | UF Volume (L) | | UF volume (L) |
| | | Tidal-7 hour + | | Tidal-7 hour + |
| Transport | UF volume (L) | 2-hour Post | UF Volume (L) | 2-hour Post |
| status | Tidal-7 hour | sorbent | Tidal-7 hour | sorbent |
| High | 0.0 | 0.0 | 0.3 | 0.4 |
| High-Average | 0.3 | 0.3 | 0.7 | 0.8 |
| Low-Average | 0.4 | 0.5 | 0.7 | 0.9 |
| Low | 0.7 | 0.8 | 1.1 | 1.4 |

### Discussion

Extending sorbent phase (i.e. the tidal therapy phase) from 7 hour (Example 1) to 9 hours (example 2) without non sorbent phase (i.e. the dwell phase) resulted in an increase in kt/V in the range of 0.21-0.52 as shown in Table 1. Similar increase in Kt/V was also estimated by extending non sorbent phase for 1 hour after sorbent phase after 7-hour sorbent phase (Table 2) or by extending non-sorbent for 2 hour after 7 hour sorbent phase (Table 3). It may be also possible to optimize the water removal by adjusting the various method of treatments in patient of different body weight and different peritoneal transport status (Example 9).

TABLE 1
Increase in Kt/V(urea) by extending
sorbent phase from 7 hour to 10 hour

| | Patient Size | | | | |
|---|---|---|---|---|---|
| | 25 L | 30 L | 40 L | 50 L | 60 L |
| Transporter Type | (45 kg) | (55 kg) | (73 kg) | (91 kg) | (109 kg) |
| High | 0.52 | 0.44 | 0.34 | 0.28 | 0.24 |
| High-Average | 0.51 | 0.43 | 0.33 | 0.27 | 0.23 |
| Low-Average | 0.47 | 0.40 | 0.31 | 0.25 | 0.21 |
| Low | 0.46 | 0.39 | 0.30 | 0.24 | 0.21 |

TABLE 2
Increase in Kt/V(urea) by having extra 1 hour
non sorbent phase after 7 hour sorbent phase

| | Patient Size | | | | |
|---|---|---|---|---|---|
| | 25 L | 30 L | 40 L | 50 L | 60 L |
| Transporter Type | (45 kg) | (55 kg) | (73 kg) | (91 kg) | (109 kg) |
| High | 0.11 | 0.09 | 0.07 | 0.06 | 0.05 |
| High-Average | 0.12 | 0.10 | 0.08 | 0.06 | 0.05 |
| Low-Average | 0.12 | 0.10 | 0.08 | 0.06 | 0.05 |
| Low | 0.13 | 0.11 | 0.08 | 0.07 | 0.06 |

TABLE 3
Increase in Kt/V(urea) by having extra 2 hours
non sorbent phase after 7 hour sorbent phase

| | Patient Size | | | | |
|---|---|---|---|---|---|
| | 25 L | 30 L | 40 L | 50 L | 60 L |
| Transporter Type | (45 kg) | (55 kg) | (73 kg) | (91 kg) | (109 kg) |
| High | 0.17 | 0.14 | 0.11 | 0.09 | 0.08 |
| High-Average | 0.20 | 0.17 | 0.13 | 0.11 | 0.09 |
| Low-Average | 0.21 | 0.18 | 0.14 | 0.11 | 0.09 |
| Low | 0.23 | 0.19 | 0.15 | 0.12 | 0.10 |

1. A method of peritoneal dialysis, the method comprising the steps of:
(i) administering to a subject a first hypertonic solution comprising a sugar to a peritoneal cavity in the subject, followed by;
(ii) a tidal therapy phase and then a dwell phase; or a dwell phase and then a tidal therapy phase;
wherein the tidal therapy phase comprises the steps of:
(a) allowing water and/or a toxin from the subject to pass into the peritoneal cavity by osmosis, thereby forming an N^{th} hypertonic solution within the peritoneal cavity;
(b) withdrawing up to about 50% by volume of the N^{th} hypertonic solution from the peritoneal cavity and combining it with a first sugar concentrate to form an N^{th}+1 hypertonic solution,
wherein the withdrawn N^{th} hypertonic solution and/or the N^{th}+1 hypertonic solution is passed through a dialysis sorbent, followed by adding calcium and magnesium ions into the withdrawn N^{th} hypertonic solution and/or the N^{th}+1 hypertonic solution;
(c) administering the N^{th}+1 hypertonic solution to the peritoneal cavity to form an N^{th}+2 hypertonic solution within the peritoneal cavity by mixing of the N^{th} and N^{th}+1 hypertonic solutions; and
(d) repeating steps (a) to (c) every 5 minutes to 30 minutes for a treatment time of about 5 hours to about 10 hours; and
wherein the dwell phase comprises the steps of:
(A) allowing water and/or a toxin from the subject to pass into the peritoneal cavity by osmosis, thereby forming an X^{th} hypertonic solution within the peritoneal cavity;
(B) withdrawing up to about 50% by volume of the X^{th} hypertonic solution from the peritoneal cavity and combining it with a second sugar concentrate to form an X^{th}+1 hypertonic solution;
(C) administering the X^{th}+1 hypertonic solution to the peritoneal cavity to form an X^{th}+2 hypertonic solution within the peritoneal cavity by mixing of the X^{th} and the X^{th}+1 hypertonic solutions;
(D) repeating steps (A) to (C) every 5 to 20 minutes for a treatment time of from about 1 to about 3 hours.

2. The method according to claim 1, wherein step (ii) comprises a tidal therapy phase and then a dwell phase, and a further tidal therapy phase following the dwell phase.

3. (canceled)

4. The method according to claim 1, wherein step (ii) comprises a dwell phase and then a tidal therapy phase, and a further dwell phase following the tidal therapy phase.

5. (canceled)

6. The method according to claim 1, wherein the first hypertonic solution has a sugar concentration of from about 1 wt % to about 4.5 wt %.

7. The method according to claim 1, wherein the sugar concentration of the N^{th} and N^{th}+1 hypertonic solutions within the peritoneal cavity during steps (a) to (d) is from about 0.8 wt % to about 4.5 wt %.

8. The method according to claim 1, wherein step (b) comprises passing the withdrawn N^{th} hypertonic solution through the dialysis sorbent followed by combining the withdrawn N^{th} hypertonic solution with the first sugar concentrate to form the N^{th}+1 hypertonic solution, wherein the first sugar concentrate comprises calcium ions and magnesium ions.

9. The method according to claim 1, wherein step (b) comprises combining the withdrawn N^{th} hypertonic solution with the first sugar concentrate to form the N^{th}+1 hypertonic solution, followed by:
passing the N^{th}+1 hypertonic solution through the dialysis sorbent, and
adding calcium and magnesium ions into the N^{th}+1 hypertonic solution after it has passed through the dialysis sorbent.

10. The method according to claim 1, wherein the calcium and magnesium ions are added into the N^{th}+1 hypertonic solution to give a calcium and magnesium ions concentration of from about 0.2 mM to about 3 mM.

11. The method according to claim 1, wherein the calcium and magnesium ions concentration of the N^{th} and N^{th}+1 hypertonic solutions within the peritoneal cavity during steps (a) to (d) is from about 0.2 mM to about 3 mM.

12. (canceled)

13. The method according to claim 1, wherein step (i) comprises administering from about 200 mL to about 4,000 mL, of the first hypertonic solution to the peritoneal cavity.

14. (canceled)

15. The method according to claim 1, wherein step (b) comprises withdrawing from about 100 mL to about 500 mL of the N^{th} hypertonic solution from the peritoneal cavity and combining the withdrawn N^{th} hypertonic solution with from about 0.1 mL to about 7 mL of the first sugar concentrate to form an N^{th}+1 hypertonic solution.

16. (canceled)

17. (canceled)

18. (canceled)

19. The method according to claim 1, wherein step (d) comprises repeating steps (a) to (c) every 7 to 17 minutes.

20. (canceled)

21. (canceled)

22. The method according to claim 1, wherein the sugar concentration of the X^{th} and X^{th}+1 hypertonic solutions within the peritoneal cavity during steps (A) to (D) is from about 1.5 wt % to about 4.5 wt %.

23. The method according to claim 1, wherein step (B) comprises withdrawing from about 100 mL to about 500 mL of the X^{th} hypertonic solution from the peritoneal cavity and combining the withdrawn X^{th} hypertonic solution with from about 0.1 mL to about 7 mL of a second sugar concentrate comprising a sugar to form an X^{th}+1 hypertonic solution.

24. (canceled)

25. (canceled)

26. (canceled)

27. The method according to claim 1, wherein step (D) comprises repeating steps (A) to (C) every 5 to 10 minutes.

28. (canceled)

29. The method according to claim 1, wherein the first and second sugar concentrates each independently have a sugar concentration of from about 0.25 g/mL to about 0.9 g/mL.

30. The method according to claim 1, wherein the sugar in the first hypertonic solution, first sugar concentrate and/or second sugar concentrate is selected from one or more of the sugars selected from the groups consisting of glucose, icodextrin and sucrose.

31. The method according to claim 1, wherein the calcium ions and/or magnesium ions present in the first sugar concentrate are derived from calcium lactate and/or magnesium lactate, respectively.

32. The method according to claim 1, wherein the second sugar concentrate is essentially free from metal ions, and the second sugar concentrate consists essentially of a sugar and water.

33. (canceled)

34. (canceled)

35. The method according to claim 1, wherein steps (i) to (ii) achieve a Kt/V(urea) of at least about 1.7.
